(19) United States
(12) Patent Application Publication
(10) Pub. No.: US 2024/0122570 A1
(43) Pub. Date: Apr. 18, 2024

(54) DEVICE AND METHOD FOR CHARACTERIZING THE EVOLUTION OF THE FLUID FLOW RATE PROFILE AT A TREATMENT AREA BY ENERGY EMISSION

(71) Applicants: EDAP TMS FRANCE, VAULX EN VELIN (FR); INSTITUT NATIONAL DE LA SANTE ET DE LA RECHERCH MEDICALE-I.N.S.E.R.M., PARIS (FR); UNIVERSITE CLAUDE BERNARD LYON I, VILLEURBANNE (FR); CENTRE LEON BERARD, LYON (FR)

(72) Inventors: Celia G. CILLEROS, VILLEURBANNE (FR); Jérémy VINCENOT, VILLEURBANNE (FR); David MELO DE LIMA, Saint Bernard (FR)

(21) Appl. No.: 18/273,560

(22) PCT Filed: Jan. 26, 2022
(86) PCT No.: PCT/FR2022/050139

§ 371 Date: Jul. 21, 2023

(30) Foreign Application Priority Data
Jan. 26, 2021 (FR) 2100694

(51) Int. Cl. A61B 8/06; A61B 8/08; A61N 7/02

(52) U.S. Cl. CPC A61B 8/06; A61B 8/488; A61N 7/02

(57) ABSTRACT

The device (1) for characterizing an evolution of the fluid flow rate profile at a treatment area includes a system (6) for controlling an energy emission probe (4) and an ultrasound imaging probe (5), configured to control the energy emission probe (4) in emission according to successive energy emission phases interspersed with energy emission stop phases, and driving the ultrasound imaging probe (5) in emission before an energy emission phase in order to measure a reference Doppler signal and during the energy emission stop phases.

### TECHNICAL FIELD

The invention relates to the technical field of apparatuses or devices including an energy emission probe, an ultrasound imaging probe and a system for controlling the emission probe and the ultrasound imaging probe. The invention finds a particularly advantageous application in the field of the application of high intensity focused ultrasound (HIFU) and the characterization of the stenosis it can cause in blood vessels.

### PRIOR ART

It is known that treatment by application of energy, such as focused ultrasound waves for example, allows to generate destruction, for example by heat, at a focal area with controlled dimensions causing irreversible lesions and destroying the targeted biological tissues. A particular application is then the treatment of tumours.

Nevertheless, this type of treatment is not without risk for the vessels, in particular blood vessels, present in the treatment area. For example, ultrasonic heating to destroy a peri-arterial tumor can lead to occlusion of the blood vessel in question. Thus, these tumors cannot be treated by application of energy without risk.

It is known to visualize by imaging the lesions caused by this energy in order to evaluate the progress of the treatment and to determine the advisability of continuing it and/or adapting it, as is in particular described in documents US 2008/0125657, US 2007/004984 and US 2003/0028111. Imaging of the treated area is performed in order to visualize the lesions caused by the treatment, but does not allow to visualize the impact of the treatment on the periphery of the treatment area. In addition, during treatment, many reactions occur in the treatment area (boiling, cavitation, necrosis, significant temperature changes, . . . ). These factors all impact the quality of an echographic acquisition.

In order to set up a safer treatment for patients, it is necessary to have a device allowing not only the emission of energy to generate the lesion of the target biological tissues, but also allowing to characterize the flow in the vessels outlying these biological tissues, preferably in real time during the treatment involving the application of energy. This would allow to limit the risk of a possible occlusion of these vessels and to control the emission of energy in order to preserve these vessels.

In this context, the Applicant sought to develop an operating cycle during which energy is successively emitted on a treatment area, then stopped for a defined period in order to evaluate the flow of fluid in a vessel in the vicinity of the treatment area, and therefore assess the impact of applying energy to this vessel in and in the vicinity of the treatment area. Thus, the objective is a characterization following an application of energy, and in particular between energy emission phases, of the modifications of the flow within vessels in the vicinity of the treatment area caused during the emission of energy, which allows to evaluate the impact of the application of energy on this same vessel at the treatment area.

Various methods are known for characterizing the flow of fluid within vessels, and in particular blood vessels. Among these, the Applicant was more particularly interested in ultrasound imaging, and in particular Doppler imaging.

Ultrasound imaging has, in fact, already been used to characterize lesions and stenoses. Doppler imaging allows, in particular, to measure the flow of fluid in a blood vessel. Ultrasound imaging has the advantage of being fast and reliable. Several types of ultrasound imaging are known and commonly used.

Conventional ultrasonic echography imaging (also called B-Mode echography) is a two-dimensional (2D) imaging that visualizes biological tissues in the area where the field of view of the ultrasound probe is positioned. This imaging therefore does not allow to evaluate the fluid flow rate in a vessel, but allows to visualize the biological tissues in an area. This imaging is therefore useful for visualizing and targeting the target biological tissues (such as a tumor for example) or the vessel to be monitored.

One-dimensional (1D) Doppler imaging provides information on the fluid flow rate in a vessel at a point or segment where the measurement area is located. This imaging therefore does not allow to visualize the surrounding biological tissues, but only the Doppler signal curve indicating the evolution of the flow rate over time of the fluid on a determined segment.

Color Doppler imaging visualizes the direction of flow in an adjustable 2D area within the echography image. The color red or blue indicates the direction of this flow. This imaging is therefore useful for visualizing the flows, in particular blood flows, in a treatment area before and after a treatment by energy emission for example, which can allow to highlight a stenosis for example.

Power Doppler imaging is based on Doppler signal energy collected in an adjustable 2D area of the echography image. This technique is more sensitive in the detection of flow and thus allows to visualize smaller vessels than color Doppler imaging but does not give information on the direction of the flow.

Among the other known types of Doppler imaging, the TM (time-motion) mode allows the study of the movements of the various structures, in particular cardiac structures, the dimensions of the cavities, in particular cardiac cavities, and the thickness of the walls.

Application US 2010/0274133 describes the detection of a stenosis of a blood vessel by Doppler measurement of the turbulent flow in this blood vessel. The Doppler probe is placed at a distance from the treatment area, downstream of the stenosis, perpendicular to the blood flow in the vessel, in order to detect the blood flow turbulence generated by the stenosis. The positioning perpendicular to the blood flow is important because it allows to measure only the turbulent flow, and to overcome the laminar flow along the blood vessel. It is then no longer possible to determine the blood flow rate in the blood vessel. In addition, the blood vessel is no longer visualized in its length, but only according to a section. The biological tissues, and in particular the tumor sought to be destroyed, can no longer be visualized. A treatment by energy emission is then difficult to conceive in parallel with this detection of stenosis.

### DISCLOSURE OF THE INVENTION

The Applicant has developed a device allowing to characterize the evolution of the flowing of the flow in the vessels at the periphery of the treatment area, caused by the emission of energy. Advantageously, the device according to the invention not only allows to visualize the modifications of the biological tissues within the treatment area, but also the modification of the rate profile in the vessels in the vicinity thereof, these modifications being caused by the emission of energy. For this purpose, the device for the characterization of an evolution in the fluid flow rate profile at the periphery of a treatment area following the emission of energy by an energy probe, thanks to an ultrasound imaging probe whose measurement area is placed outside the treatment area, comprises a circuit for processing the received Doppler signal, the ultrasound imaging probe being positioned to produce an image plane in which the flow of fluid is observed along its longitudinal axis, said device including a system for controlling the energy emission probe and the ultrasound imaging probe, configured to:
control the energy emission probe in emission according to successive energy emission phases interspersed with energy emission stop phases, said energy emission stop phases having a duration ranging from 1.5 s to 15 s and preferably from 1.5 s to 2.5 s,
drive the ultrasound imaging probe in emission before an energy emission phase in order to measure a reference Doppler signal and during the energy emission stop phases to measure a received Doppler signal after each energy emission phase),
and allow to provide monitoring of the evolution of the fluid flow rate profile resulting from the ultrasound imaging.

This device allows to measure the flow rate of the flow using the ultrasound imaging probe between each energy emission phase, and thus to determine the evolution of this flow over time, and more particularly in the vessels close to the treatment area. The curve of the Doppler signal provides an indication of the impact of the application of energy on the vessel, which allows to assess the state of health of the vessel, or even to anticipate an occlusion thereof.

The device according to the invention may also have either one of the following features, or any combination of two or more of these features:
the ultrasound imaging probe gives access to at least one Doppler curve mode, such as the 1D Doppler mode or the TM mode, to measure an evolution in the fluid flow rate profile at the measurement area, and at least one two-dimensional imaging mode, such as the B mode or the color Doppler mode, to visualize biological tissues of the treatment area,
the control system drives the energy emission probe in emission according to successive energy emission phases interspersed with energy emission stop phases, according to a duty cycle comprised between 30% energy emission phase/70% energy emission stop phase and 90% energy emission phase/10% energy emission stop phase
the Doppler signal processing circuit analyzes the evolution of the rate profile obtained by Doppler effect,
the Doppler signal processing circuit includes a system for comparing the received Doppler signal curve and the reference Doppler signal curve after each energy emission phase, to determine the evolution of the rate profile obtained by the ultrasound imaging,
the comparison system of the Doppler signal processing circuit carries out the comparison between an average of 3 to 5 received Doppler signal curves and an average of 3 to 5 reference Doppler signal curves,
the Doppler signal processing circuit determines the evolution of the rate profile thanks to the difference between the received Doppler signal(s) curve(s) and the reference Doppler signal(s) curve(s),
the Doppler signal processing circuit determines the evolution of the rate profile thanks to the derivative of the difference between the received Doppler signal(s) curve(s) and the reference Doppler signal(s) curve(s),
the Doppler signal processing circuit includes a system for comparing with a threshold value the received Doppler signal curve(s), the difference between the received Doppler signal(s) curve(s) and the reference Doppler signal(s) curve(s), or the derivative of the difference between the received Doppler signal(s) curve(s) and the reference Doppler signal(s) curve(s),
the processing circuit includes a cut-off system stopping the energy emission probe when the received Doppler signal curve(s), the value of the difference between the received Doppler signal(s) curve(s) and the reference Doppler signal(s) curve(s), or the value of the derivative of the difference between the received Doppler signal(s) curve(s) and the reference Doppler signal(s) curve(s) reaches said threshold value,
the device includes a database recording the received Doppler signal curves, the difference between the received Doppler signal(s) curve(s) and the reference Doppler signal(s) curve(s), and/or the value of the derivative of the difference between the received Doppler signal(s) curve(s) and the reference Doppler signal(s) curve(s) in order to determine the threshold value by itself,
the energy emission probe is a focused ultrasound wave emission probe,
the energy emission probe is an electroporation emission probe, a radio frequency emission probe or a microwave emission probe.

The invention also relates to a method for characterizing an evolution of the fluid flow rate profile close to a treatment area following an emission of energy using the device according to the invention, and comprising the following steps:
a—positioning the measurement area of the ultrasound imaging probe outside the treatment area, 5 to 60 mm from the treatment area, and longitudinally relative to the fluid flow axis,
b—driving the ultrasound imaging probe in emission before an energy emission phase of an energy emission probe in order to measure a reference Doppler signal curve and during energy emission stop phases, interspersing the energy emission phases for a duration comprised between 1.5 s and 15 s, preferably between 1.5 s and 2.5 s, to measure a received Doppler signal curve after each energy emission phase.

The method according to the invention then allows to evaluate during the treatment by energy emission, and advantageously in real time, the lesions caused on the biological tissues by the emission of energy in the treatment area, as well as the evolution of the rate profile of the fluid flow in a vessel in the vicinity of the treatment area. The energy emission therapy is then safer and more effective, as the practitioner can assess the impact of the energy emission therapy over time and determine when it is more appropriate to stop the energy emission therapy, not only according to the lesions caused to the biological tissues but also according to the evolution of the flow of fluid in the vessel.

Indeed, in the context of the invention, ultrasound imaging, and in particular Doppler imaging, allows to have an indication over time, and preferably in real time, of the impact of the application of energy not only on the biological tissues, and in particular comprised in the field of view of the ultrasound imaging probe, but also on the vessel, and in particular at the measurement area of the ultrasound imaging probe, which allows to evaluate the state of health of the vessel during and after the emission of energy, or even to anticipate an occlusion of said vessel, and therefore to control the emission of energy in order to preserve the vessel within and in the vicinity of the treatment area. The method according to the invention is then safer for the patient since it allows in particular to stop the treatment by energy emission before the stenosis of the vessel, even before or at the time of the start of the stenosis process.

The method according to the invention may also have either one of the following features, or any combination of two or more of these features:
the measurement area of the ultrasound imaging probe is positioned upstream of the treatment area,
the received Doppler signal curve(s) is/are compared with the reference Doppler signal,
the evolution of the rate profile is determined thanks to the derivative of the difference between the received Doppler signal curve(s) and the reference Doppler signal curve(s),
the stop of the emission of the energy emission probe is driven when the received Doppler signal curve exceeds a threshold value,
the energy emission probe is driven in emission according to successive energy emission phases interspersed with energy emission stop phases, according to a duty cycle ranging from 30% energy emission phase/70% energy emission stop phase to 90% energy emission phase/10% energy emission stop phase,
the duration of each energy emission phase and/or the duration of each energy emission stop phase varies/vary,
the energy emission probe is a focused ultrasound wave emission probe,
the treatment area is located at the heart, the pancreas, the liver, the kidneys, or the blood-brain barrier, and the ultrasound imaging probe is located on a blood vessel.

### BRIEF DESCRIPTION OF THE DRAWINGS

FIG. 1 is a diagram of an exemplary embodiment of a treatment device in accordance with the invention represented in a simplified manner during the treatment of biological tissues.

FIG. 2 is a diagram representing in a simplified manner the field of view of the ultrasound imaging probe, including the treatment area by energy emission and the measurement area of the ultrasound imaging probe.

FIG. 3 is a diagram representing the method for using the device according to the invention.

FIG. 4 represents an example of profile of curves obtained (A) before treatment, (B) to (D) during treatment and (E) at the end of treatment by HIFU of the hepatic artery of a porcine model.

FIG. 5 represents the evolution over time of the blood flow rate profile in the hepatic artery of a porcine model (curve (1): difference, curve (2): derivative of the difference, line (S): threshold value defined for the studied model).

### DESCRIPTION OF THE EMBODIMENTS

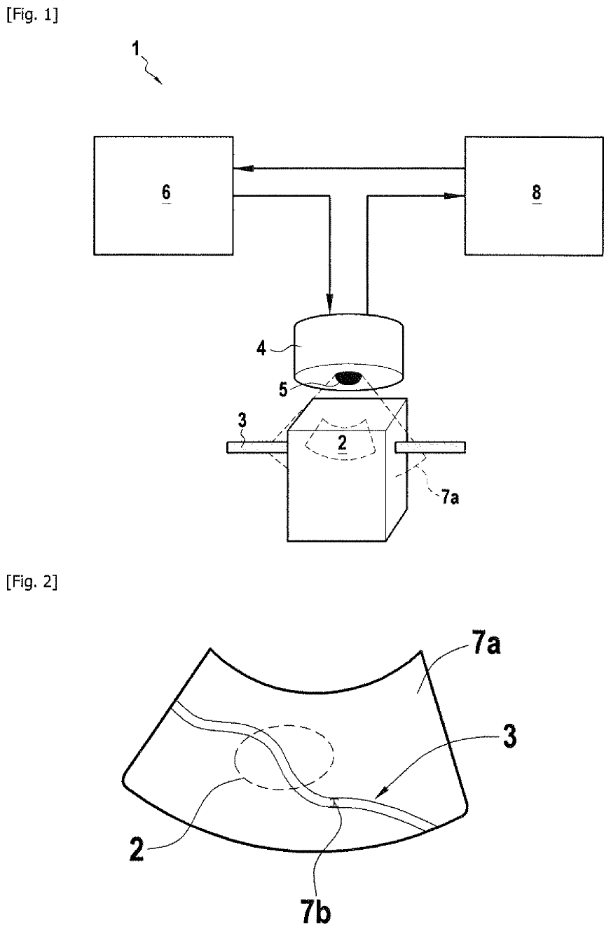

As illustrated in FIG. 1, the invention relates to a device 1 for characterizing an evolution in the fluid flow rate profile at the periphery of the treatment area 2 following an emission of energy.

In the context of the invention, “close to”, “at the periphery of” and “in the vicinity of” the treatment area 2 are used interchangeably and mean located at a distance from the treatment area 2, that is to say not included in the treatment area 2 but located near the treatment area 2, and in particular at a distance of less than 60 mm, typically at a distance ranging from 5 mm to 60 mm from the treatment area 2, and preferably from 8 mm to 15 mm.

The treatment area 2 comprises biological tissues of a living being. The targeted biological tissues are typically tumours, the resection of which by surgical means could be difficult or even impossible, and which can be treated thanks to an energy emission. The treatment area 2 includes at least one vessel 3 for the flow of fluid, typically a blood vessel, on which lesions can be caused by the energy emitted by an energy emission probe 4.

According to the embodiment illustrated in FIG. 1, the device 1 includes an energy emission probe 4, an ultrasound imaging probe 5 and a system 6 for controlling the energy emission probe 4 and the ultrasound imaging probe 5. Although preferred, this embodiment is not limiting, and it could be considered that the device 1 according to the invention includes a control system configured to drive an energy emission probe 4 and to drive an ultrasound imaging probe 5, both of them, or only one of the two, being distinct from the device 1 according to the invention and adapted to be connected to the latter. In the following description, the preferred embodiment where the device includes an energy emission probe 4, an ultrasound imaging probe 5 and a system 6 for controlling the energy emission probe 4 and the ultrasound imaging probe 5 is more particularly detailed, but what is explained below applies in the same way to a device not including an energy emission probe 4 and/or an ultrasound imaging probe 5, but being intended to be connected thereto, without departing from the scope of the invention.

The energy emission probe 4 can be of any type known per se for the treatment of biological tissues, such as an electroporation, radio frequency, microwave or focused ultrasound wave emission probe. According to a preferred embodiment of the invention, the energy emission probe 4 is a focused ultrasound wave emission probe, or High Intensity Focused Ultrasound (HIFU). In the latter case, in the usual manner, the probe for emitting focused ultrasound waves comprises a transducer including ultrasound emitters.

The control system 6 represents all the control and emission elements. This can be, for example, a signal generator for therapy and/or a signal generator for imaging and/or a computer.

The energy emission probe 4 is driven in emission by the control system 6. In other words, the control system 6 causes the emission and the stop of the emission of energy by the energy emission probe 4. Thus, the control system 6 controls alternating energy emission phases A1 and energy emission stop phases B1. The duration of these successive energy emission A1 and energy emission stop B1 phases can be chosen by the user. The duration and intensity of the energy emission phases A1 depend on the treatment area 2, the target biological tissue and the features of the vessels present in and in the vicinity of the treatment area, and typically have a duration ranging from 1 s to 120 s. The energy emission stop phases B1 interspersing the energy emission phases A1 can also be chosen by the user and typically have a duration ranging from 1.5 s to 15 s, preferably 1.5 s to 10 s, more preferably from 1.5 s to 2.5 s. These successive energy emission A1 and energy emission stop B1 phases may have their durations which vary independently of each other.

When the energy emission probe 4 is a focused ultrasound wave emission probe, the control system 6 comprises a generator capable of activating or stopping the operation of the ultrasound emitters.

In the context of the invention, the ultrasound imaging probe 5 can be used according to different modes. According to a particular embodiment, the ultrasound imaging probe 5 is a Doppler imaging probe.

In the context of the invention, the ultrasound imaging probe 5 is used according to at least one two-dimensional imaging mode, preferably one or two 2D imaging mode(s) and in particular two 2D imaging modes, for example in color Doppler mode and/or in B mode. The use of this mode allows to visualize the biological tissues in the field of view 7a of the ultrasound imaging probe, which includes the treatment area 2 as shown in FIG. 1 or in FIG. 2. The choice of the two-dimensional mode(s) of the ultrasound imaging probe depends on the target tissues and the patient being treated, and can be determined by the user. Thus, this 2D mode allows to visualize the biological tissues, and in particular the treatment area 2 and optionally its periphery, following an emission of energy by the energy emission probe 4, which also allows the targeting of the emission of energy.

In the context of the invention, the ultrasound imaging probe 5 is also used according to at least one one-dimensional (1D) mode or curved Doppler mode, such as the TM mode or the power Doppler mode, such as for example the Power Doppler probes used during a cardiac examination. As shown in the shape of a segment in FIG. 2, the measurement area 7b of the ultrasound energy probe in Doppler curve mode is placed outside the treatment area 2: the measurement area 7b is positioned on a vessel passing through the treatment area 2, upstream or downstream thereof with respect to the flow of fluid. Preferably, the measurement area 7b of the ultrasound imaging probe 5 is placed upstream of the treatment area 2 with respect to the flow of the fluid. The ultrasound imaging probe in Doppler curve mode thus allows to measure the fluid flow rate profile in a vessel upstream or downstream of the treatment area 2, such as for example the flow of blood in a blood vessel. By extrapolation, this measurement gives an indication of the flow of fluid within the treatment area 2, and therefore of the state of health of the vessel in the treatment area 2.

In the context of the invention, the field of view 7a of the ultrasound imaging probe allows to produce an image plane in which the flow of fluid is observed along its longitudinal axis. In the context of the invention, the “longitudinal” axis of the fluid flow means the axis in the direction of the length of the vessel through which the fluid flows. It therefore differs from the plane perpendicular to the flow of fluid used in the prior art.

According to a preferred embodiment, as illustrated in FIG. 1, the energy emission probe 4 and the ultrasound imaging probe 5 are aligned. For example, the energy emission probe 4 can be a focused ultrasound probe integrating in its center an ultrasound imaging probe 5 so that their respective acoustic axis coincides. This embodiment is however not limiting, and the device may be such that these two probes are not aligned.

According to the particular embodiment illustrated in FIG. 1, the energy emission probe 4 is made in the form of a box having an emission face at the center of which the ultrasound imaging probe 5 is positioned. The ultrasound imaging probe 5 and the energy emission probe 4 are mounted together. Any other positioning could nevertheless be considered without departing from the scope of the invention.

The ultrasound imaging probe 5 is driven by the control system 6, in emission and optionally in emission stop. More precisely, the control system 6 causes the emission of the ultrasound imaging probe 5, and optionally the stop of the emission of the ultrasound imaging probe for all or part of the energy emission stop phases B1.

According to a first embodiment, the ultrasound imaging probe 5 is controlled by the control system 6 only in emission, that is to say that the control system 6 controls the ultrasound imaging probe 5 in continuous emission. According to a second embodiment, the ultrasound imaging probe 5 is controlled by the control system 6, during all or part of the energy emission stop phases B1, in emission as well as in emission stop. According to this second embodiment, the emission of ultrasound energy is not continuous.

Regardless of the embodiment, during the stop phases of the emission of the energy B1, the received Doppler signal is measured. This measured Doppler signal is preferably recorded during this same energy emission stop phase B1, or can be recorded during this same energy emission stop phase and during the next energy emission phase. The received Doppler signal is understood as the Doppler signal curve obtained using the ultrasound imaging probe used in 1D mode (that is to say Doppler curve mode) and the echographic plane obtained using the ultrasound imaging used in 2D mode.

In summary, the device 1 includes a system 6 for controlling the energy emission probe 4 and the ultrasound imaging probe 5 which alternately controls the energy emission probe 4 (in energy emission and emission stop) and the ultrasound imaging probe 5 (in emission and optionally in emission stop of the Doppler signal, during all or part of the energy emission stop phases). Preferably, the successive energy emission phases A1 and energy emission stop phases B1 are according to a duty cycle comprised between 30% energy emission phase/70% energy emission stop phase and 90% energy emission phase/10% energy emission stop phase, preferably 70% energy emission phase/30% energy emission stop phase.

The ultrasound imaging probe 5 usually comprises a transducer including ultrasound emitters and ultrasound receivers, as well as a circuit 8 for processing the received Doppler signal.

The ultrasound emitters are advantageously pulsed Doppler emitters.

The processing circuit 8 represents all the reception and analysis elements. This can, for example, be a signal analyzer for therapy and/or a signal analyzer for imaging and/or a computer. The processing circuit 8 of the received Doppler signal allows to record the received Doppler signal curves. In particular, a received Doppler signal curve, called reference Doppler signal curve, is measured before an energy emission phase A1 and recorded. This reference Doppler signal curve may be the first measured received Doppler signal curve, or a subsequently received Doppler signal curve. The reference Doppler signal curve can therefore be selected from the set of Doppler signal curves acquired prior to the considered received Doppler signal curve. Preferably, the reference Doppler signal curve is the Doppler signal curve measured before the first energy emission phase A1. In this case, the control system 6 initially drives the energy emission probe 4 to stop, according to a first energy emission stop phase B1 during which the reference Doppler signal curve is measured by the processing circuit 8, then drives the energy emission probe 4 in emission for a first energy emission phase A1. Then, stop and energy emission phases follow each other successively.

The device 1 according to the invention allows to analyze the evolution of the received Doppler signal curve, and therefore the evolution of the rate profile of the fluid flow in the vicinity of the treatment area 2. For this purpose, the processing circuit 8 of the ultrasound imaging probe 5 includes a system for comparing the received Doppler signal curve with the reference Doppler signal curve. Thus, after each energy emission phase A1, and preferably during each energy emission stop phase B1, the received Doppler signal curve is recorded and compared with the reference Doppler signal curve. This comparison allows to identify differences between the received Doppler signal curve and the reference Doppler signal curve, and thus to determine the evolution of the rate profile of the fluid flow.

The comparison between the received Doppler signal curve and the reference Doppler signal curve can be carried out between several Doppler signal curves, and in particular between 3 to 5 curves. Preferably, the comparison is made between an average of 3 to 5 received Doppler signal curves and an average of 3 to 5 reference Doppler signal curves.

The comparison between the received Doppler signal curve and the reference Doppler signal curve can be made thanks to the difference between the received Doppler signal(s) curve(s) and the reference Doppler signal(s) curve(s). The derivative of the difference between the received Doppler signal(s) curve(s) and the reference Doppler signal(s) curve(s) can further be calculated. The study of the derivative makes it easier to identify a change in fluid flow behavior (rate, nature of the flow, for example appearance of a vortex component of the flow).

According to one embodiment, a threshold value can be recorded by the processing system 8 in order to be compared with the received Doppler signal curve. Thus, when the received Doppler signal reaches this threshold value, the user can be notified by a warning system, for example by a light signal or an audible signal. It could also be considered that the control system 6 causes the energy emission to stop when the threshold value is reached thanks to a cut-off system. Thus, when this threshold value represents for example a value from which the risk of stenosis is high, the practitioner is warned and can act in order to avoid or limit the risk of stenosis, and thus best preserve the health of the person treated using the device 1 according to the invention.

This threshold value can for example be a difference value between the received Doppler signal curve and the reference Doppler signal curve, or a value of the derivative of this difference. This threshold value depends on the target biological tissues and on the patient, and can be determined by the user of the device 1, or be determined thanks to a database which records the received Doppler signal curves. In the latter case, the device 1 is capable of determining the threshold value by itself thanks to the database that it includes. This database can in particular record the received Doppler signal curves for the same patient during different treatments for example, or else for several patients, for the same treatment area.

The invention also relates to a method for determining the evolution of the flow in a vessel upstream or downstream of the treatment area and caused by the prior emission of energy during the use of the device 1 according to the invention. In the context of the invention, the emission of energy causes a modification of the fluid flow rate profile within and close to the treatment area 2, and the method according to the invention allows, by determining the evolution of this rate profile over time, and preferably in real time, to estimate the impact on the blood vessel within and at the periphery of the treatment area of the prior emission of energy, and more precisely after each emission of energy.

In the context of the invention, “in real time” means in a short or minimal time interval with respect to the moment when the event takes place, and typically less than 15 s, preferably less than 10 s, or even less than 5s, and ideally less than 2s.

The device 1 is as detailed above. In particular, the energy emission probe 4 can be of any type known per se for the emission of energy on biological tissues, such as an electroporation, radiofrequency, microwave or, preferably, focused ultrasound wave emission probe. The ultrasound imaging probe 5 is used according to at least one one-dimensional mode (that is to say Doppler curve mode), such as power Doppler mode and/or TM mode, and also according to at least one two-dimensional imaging mode, preferably one or two imaging modes, such as for example the color Doppler mode and/or the B mode. Thus, the method according to the invention not only allows to visualize the biological tissues, but also the fluid flow rate at the periphery of the treatment area 2 by emission of energy.

The method according to the invention comprises a first step a) during which the measurement area 7b of the ultrasound imaging probe 5 (in Doppler curve mode) is placed outside the treatment area 2, at a distance ranging from 5 mm to 60 mm from the treatment area 2, and preferably from 8 mm to 15 mm, and longitudinally relative to the fluid flow axis the rate profile of which is studied. The measurement area 7b can be placed upstream or downstream of the treatment area 2 with respect to the fluid flow, preferably upstream of the treatment area 2, and in particular at a distance ranging from 5 mm to 60 mm, preferably from 8 mm to 15 mm. When the measurement area 7b is placed downstream, it is preferably placed at a distance ranging from 10 mm to 50 mm from the treatment area 2.

Preferably also during this step a), the field of view 7a of the ultrasound imaging probe 5 (in 2D imaging mode) is positioned so as to cover the treatment area 2 and the measurement area 7b, as illustrated in FIG. 2.

The energy emission probe 4 is controlled in emission by the control system 6, according to successive energy emission phases A1 and energy emission stop phases B1, as described above in connection with the device 1 according to the invention.

In the usual way for the person skilled in the art, the energy emission probe 4 is positioned close to, in contact with or even within the treatment area 2, depending on the nature of the energy emission probe used.

According to an advantageous embodiment, and in particular in the case where the energy emission probe is a HIFU probe, the energy emission probe 4 and the ultrasound imaging probe 5 are positioned so that the field of view 7a of the ultrasound imaging probe 5 and that of the energy emission probe 4 are aligned. This can be achieved for example by integrating the ultrasound imaging probe 5 in the center of the energy emission probe 4. This embodiment is not limiting, and it may be considered not to align the field of view 7a of the ultrasound imaging probe 5 and that of the energy emission probe 4 without departing from the scope of the invention.

The method also comprises a step b) during which the ultrasound imaging probe 5 is driven in emission, and possibly in emission stop during the energy emission phases by the control system 6. The reference Doppler signal curve (before an energy emission phase A1) as well as the received Doppler signal curve (during the energy emission stop phases B1), as previously defined, are measured during this step. These Doppler signals are measured during the energy emission stop phases B1 and recorded during said energy emission stop phases B1 and/or during the subsequent energy emission phases.

Advantageously, the durations of the energy emission phases A1 and of the successive energy emission stop phases B1 are according to a ratio ranging from 30% energy emission phase/70% energy emission stop phase to 90% energy emission phase/10% energy emission stop phase, and preferably 70% energy emission phase/30% energy emission stop phase.

It may be considered to start the method according to the invention with an energy emission phase A1 or else to start with an energy emission stop phase B1 during which the Doppler signals are emitted and received. In the latter case, the reference Doppler signal curve is preferably measured during this first energy emission stop phase B1.

The number of energy emission A1 and energy emission stop B1 phases are determined by the user. Several parameters can be taken into account by the user to determine the number of energy emission A1 and energy emission stop B1 phases. In the case of the treatment of a tumor, the features of the tumor (type, size, organ where the tumor is located), of the vessel (diameter, flow rate and biological features) as well as the type of energy emission probe used may in particular be taken into account.

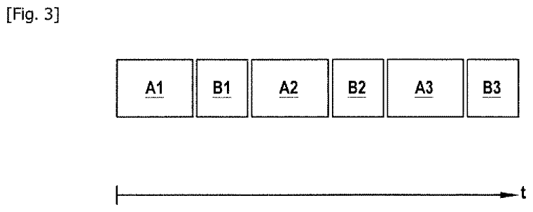

This alternation of energy emission phases A1 and of energy emission stop phases B1, during which the Doppler signal is measured and recorded, is illustrated in FIG. 3. In the embodiment illustrated in FIG. 3, the energy emission probe 4 is a HIFU probe, the durations of energy emission and Doppler signal emission are the same in each phase, and the energy emission phase/energy emission stop phase duration ratio is 60/40. According to this illustrated embodiment, three energy emission phases (A1, A2 and A3) are interspersed with three energy emission stop phases (B1, B2 and B3). The illustrated method begins with an energy emission phase. This embodiment is nevertheless not limiting, and any other energy emission probe, any other duration and number of energy emission and energy emission stop phases could be considered without departing of the scope of the invention.

In particular, it may be considered to vary the duration of the energy emission phases and/or of the energy emission stop phases over time. Thus, according to a particular embodiment, the energy emission phases A1 are increasingly shorter as the treatment progresses, and the energy emission stop phases B1 are increasingly longer. It may also be considered according to another embodiment to vary these durations in a non-linear manner.

In order to characterize the modifications in fluid the flow rate in the vessel 3, the received Doppler signal(s) curve(s) is (are) advantageously compared to the reference Doppler signal curve. As explained above, each (received and/or reference) Doppler signal curve can be calculated by performing an average of several measured Doppler signal curves, and in particular 3 to 5 Doppler signal curves. As detailed above, the comparison can be made using the difference between the curve(s) of the received and reference Doppler signal(s), or using the derivative of this difference.

The measurement of the received Doppler signal is carried out, at regular time intervals or not, at each energy emission stop phase B1. Thus, the measurement of the received Doppler signal curve and its comparison with the reference Doppler signal curve allow to follow the evolution of the flow of fluid progressively, and advantageously in real time. Then, this allows the practitioner to follow the evolution of the impact of the energy emission on the studied vessel is present in the treatment area 2.

With the aim of following even more easily and even more safely for the patient the evolution of the rate profile of the flow of fluid caused by the emission of energy, it is possible that the processing circuit 8 includes a system for comparing the received Doppler signal curve with a threshold value, as described previously. Thus, as soon as this threshold value is reached, the energy emission probe 4 can automatically be stopped by a cut-off system.

The method according to the invention advantageously allows to treat a wide variety of biological tissues, among which may be mentioned the heart, the pancreas, the liver, the kidneys, or the blood-brain barrier. In this case, the ultrasound imaging probe advantageously allows to measure the Doppler signal of a blood vessel.

The device according to the invention advantageously allows to carry out ablative therapies, and in particular the ablation of tumors, in a non-invasive and safer manner for the patient since the risk of stenosis is limited or even eliminated.

### EXAMPLES

The device according to the invention was used to treat the pancreas of a porcine model. The measurement area is located on the hepatic artery, upstream of the treatment area.

The emission of energy is carried out thanks to a HIFU probe.

The HIFU probe is driven in emission according to energy emission phases of s each, for 36 energy emission phases (for a total HIFU emission of 360 s).

The sequence begins with an energy emission phase, followed by an energy emission stop phase. The HIFU probe is driven in energy emission stop (B1) during the ultrasound imaging phases lasting 15 seconds, and in energy emission during the energy emission phases (A1). The ultrasound imaging probe is driven in signal acquisition stop during the energy emission phases (A1) and in signal acquisition during part (2 seconds) of the energy emission stop phases (B1).

The ultrasound imaging probe is a Doppler imaging probe (1D and 2D) used in power Doppler mode and in combined B mode/color Doppler mode.

The Doppler imaging probe and the HIFU probe are aligned.

The algorithm analyzes 1D Doppler signal curves. The time curve and its evolution during processing are interpreted by the calculation algorithm. The entire signal is used.

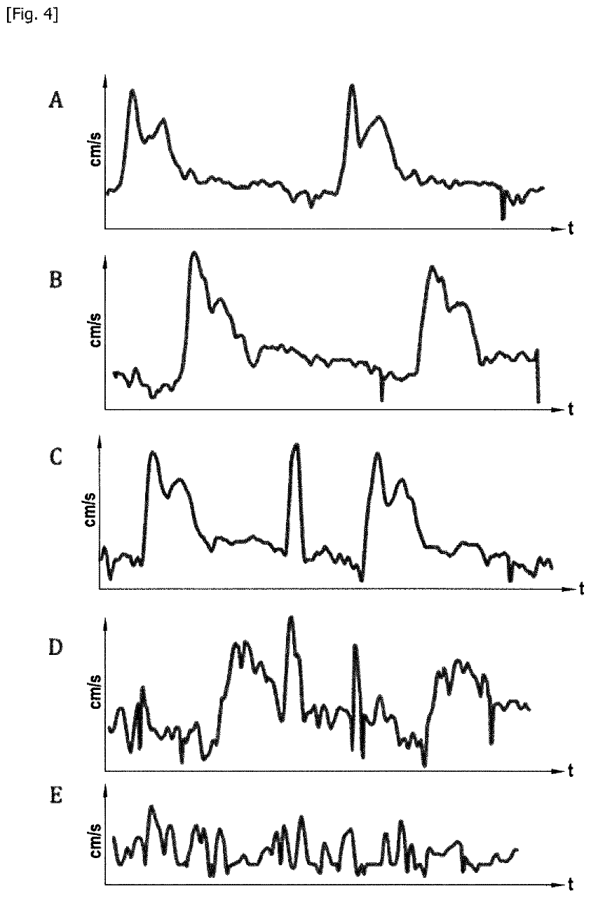

FIG. 4 represents the evolution of the signal changes over time compared to a reference Doppler signal curve (signal A). Signals A to E correspond to Doppler signal curves acquired at the start (signal A), during (signals B to D) or at the end of treatment (signal E).

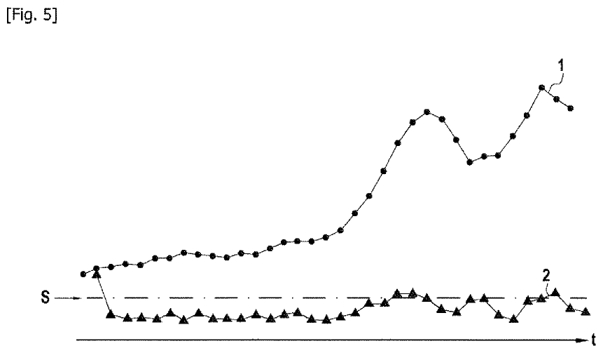

A curve representing the evolution of the difference in the rate profile during the treatment according to FIG. 4 is obtained by averaging five difference values (FIG. 5). Curve (1) represents the evolution in the difference between the received Doppler signal curve and the reference Doppler signal curve, over an average of 5 measurements. Curve (2) plots the slope of the difference curve (1). It therefore corresponds to the derivative of the difference. Line S determines the threshold value supposed to define the limit that the slope must not exceed to ensure a treatment avoiding the loss of the arterial signal. In the example shown, the threshold value is set to 1.

The more the signals are decorrelated, the greater the difference. The progressive decorrelation of the signals over time results in a steady increase in the difference curve (1). If the difference is too large and increases rapidly, then the slope (2) tends to exceed this threshold because the signals become extremely disturbed compared to the reference signals.

1. A device (1) for characterizing an evolution in the fluid flow rate profile at the periphery of a treatment area (2), following the emission of energy by an energy emission probe (4), thanks to an ultrasound imaging probe (5) whose measurement area (7b) is placed outside the treatment area (2), said ultrasound imaging probe (5) comprising a circuit (8) for processing the received Doppler signal, the ultrasound imaging probe (5) being positioned to produce an image plane in which the flow of fluid is observed along its longitudinal axis, said device (1) including a system (6) for controlling the energy emission probe (4) and the ultrasound imaging probe (5), configured to:
control the energy emission probe (4) in emission according to successive energy emission phases (A1) interspersed with energy emission stop phases (B1), said energy emission stop phases (B1) having a duration ranging from 1.5 s to 15 s and preferably from 1.5 s to 2.5 s,
drive the ultrasound imaging probe (5) in emission before an energy emission phase (A1) in order to measure a reference Doppler signal and during the energy emission stop phases (B1) to measure a received Doppler signal after each energy emission phase (A1),
and allow to provide monitoring of the evolution of the fluid flow rate profile resulting from the ultrasound imaging.

2. The device (1) according to claim 1, wherein the ultrasound imaging probe (5) gives access to at least one Doppler curve mode, such as the 1D Doppler mode or the TM mode, to measure an evolution in the fluid flow rate profile at the measurement area (7b), and at least one two-dimensional imaging mode, such as the B mode or the color Doppler mode, to visualize biological tissues of the treatment area (2).

3. The device according to claim 1, wherein the control system (6) controls the energy emission probe (4) in emission according to successive energy emission phases (A1) interspersed with energy emission stop phases (B1), according to a duty cycle comprised between 30% energy emission phase/70% energy emission stop phase and 90% energy emission phase/10% energy emission stop phase.

4. The device (1) according to claim 1, wherein the Doppler signal processing circuit (8) analyzes the evolution of the rate profile obtained by Doppler effect.

5. The device (1) according to claim 2, wherein the Doppler signal processing circuit (8) includes a system for comparing the received Doppler signal curve and the reference Doppler signal curve after each energy emission phase (AI), to determine the evolution of the rate profile obtained by the ultrasound imaging.

6. The device (1) according to claim 5 according to which the comparison system of the Doppler signal processing circuit (8) carries out the comparison between an average of 3 to 5 received Doppler signal curves and an average of 3 to 5 reference Doppler signal curves.

7. The device (1) according to claim 5, according to which the Doppler signal processing circuit (8) determines the evolution of the rate profile thanks to the difference between the received Doppler signal(s) curve(s) and the reference Doppler signal(s) curve(s).

8. The device (1) according to claim 5, according to which the Doppler signal processing circuit (8) determines the evolution of the rate profile thanks to the derivative of the difference between the received Doppler signal(s) curve(s) and the reference Doppler signal(s) curve(s).

9. The device (1) according to claim 2, wherein the Doppler signal processing circuit includes a system for comparing with a threshold value the received Doppler signal curve(s), the difference between the received Doppler signal(s) curve(s) and the reference Doppler signal(s) curve(s), or the derivative of the difference between the received Doppler signal(s) curve(s) and the reference Doppler signal(s) curve(s).

10. The device according to claim 9, according to which the processing circuit (8) includes a cut-off system stopping the energy emission probe (4) when the received Doppler signal(s) curve(s), the value of the difference between the received Doppler signal(s) curve(s) and the reference Doppler signal(s) curve(s), or the value of the derivative of the difference between the received Doppler signal(s) curve(s) and the reference Doppler signal(s) curve(s) reaches said threshold value.

11. The device (1) according to claim 9 including a database recording the received Doppler signal curves, the difference between the received Doppler signal(s) curve(s) and the reference Doppler signal(s) curve(s), and/or the value of the derivative of the difference between the received Doppler signal(s) curve(s) and the reference Doppler signal(s) curve(s) in order to determine the threshold value by itself.

12. The device (1) according to claim 1, wherein the energy emission probe (4) is a focused ultrasound wave emission probe.

13. The device (1) according to claim 1 wherein the energy emission probe (4) is an electroporation emission probe, radio frequency emission probe or a microwave emission probe.

14. A method for characterizing an evolution of the fluid flow rate profile at the periphery of a treatment area following the emission of energy using the device (1) according to claim 1, comprising the following steps:
a—positioning the measurement area (7b) of the ultrasound imaging probe (5) outside the treatment area (2), 5 to 60 mm from the treatment area (2), and longitudinally relative to the fluid flow axis,
b—driving the ultrasound imaging probe (5) in emission before an energy emission phase (A1) of an energy emission probe (4) in order to measure a reference Doppler signal curve and during energy emission stop phases (B1), interspersing the energy emission phases (A1) for a duration comprised between 1.5 s and 15 s, preferably between 1.5 s and 2.5 s, to measure a received Doppler signal curve after each energy emission phase (A1).

15. The method according to claim 14, wherein the measurement area (7b) of the ultrasound imaging probe (5) is positioned upstream of the treatment area (2).

16. The method according to claim 14 wherein the received Doppler signal curve(s) is/are compared with the reference Doppler signal.

17. The method according to claim 16, wherein the evolution of the rate profile is determined thanks to the derivative of the difference between the received Doppler signal curve(s) and the reference Doppler signal curve(s).

18. The method according to claim 14, according to which the stop of the emission of the energy emission probe (4) is driven when the received Doppler signal curve exceeds a threshold value.

19. The method according to claim 14, according to which the energy emission probe (4) is driven in emission according to successive energy emission phases (A1) interspersed with energy emission stop phases (B1), according to a duty cycle ranging from 30% energy emission phase (A1)/70% energy emission stop phase (B1) to 90% energy emission phase (A1)/10% energy emission stop phase (B1).

20. The method according to claim 14, according to which the duration of each energy emission phase (A1) and/or the duration of each energy emission stop phase (B1) varies/vary.

21. The method according to claim 14, wherein the energy emission probe (4) is a focused ultrasound wave emission probe.

22. The method according to claim 14, wherein the treatment area (2) is located at the heart, the pancreas, the liver, the kidneys, or the blood-brain barrier, and the ultrasound imaging probe (5) is located on a blood vessel.
